(19) United States
(12) Patent Application Publication
(10) Pub. No.: US 2012/0027812 A1
(43) Pub. Date: Feb. 2, 2012

(54) INTRAOCULAR LENSES TREATED WITH ALKYLPHOSPHOCHOLINES FOR PHARMACOLOGICAL AFTERCATARACT PROPHYLAXIS

(76) Inventors: Kirsten Eibl-Lindner, Munchen (DE); Anselm Kampik, Munchen (DE); Hansjörg Eibl, Bovenden (DE)

(21) Appl. No.: 13/148,127

(22) PCT Filed: Feb. 8, 2010
(86) PCT No.: PCT/EP2010/051490

§ 371 Date: Oct. 19, 2011

(30) Foreign Application Priority Data
Feb. 6, 2009 (DE) 10 2009 007 853.3

(51) Int. Cl. A61K 31/685; A61P 27/12; A61P 27/02; C07F 9/09; A61K 9/00

(52) U.S. Cl. 424/400; 558/166; 514/114

(57) ABSTRACT

The invention relates to ophthalmological implants which comprise alkylphosphocholines. Implants of this type may in particular advantageously be used for the prophylaxis of aftercataract.

The invention relates to medical materials, and in particular ophthalmological implants, which comprise alkylphosphocholines (APC). Implants of this type may in particular advantageously be used for the prophylaxis of aftercataract.

The cataract is the most common cause of avoidable blindness in old age and/or in connection with diabetes mellitus (Klein et al.; Ophthalmology 1984; 91: 1-9) worldwide (48%; WHO Global Initiative to Eliminate Avoidable Blindness, “Vision 2020: The Right to Sight”). It takes the form of an opacification of the ocular lens, and this results in a reduction in the transmission of light through the eye onto the retina (media opacification). If the retina is intact, the patient can regain high visual acuity immediately by way of a relatively simple operation involving removal of the opacified lens along with implantation of an artificial lens (intraocular lens; IOL) in the capsular bag which is left behind.

According to estimates in the industry, approximately 520,000 cataract operations a year are performed in Germany (see “Derzeitiger Stand der Katarakt-und refraktiven Chirurgie-Ergebnisse der Deutschen Gesellschaft für Intraokularlinsenimplantation, Interventionelle und Refraktive Chirurgie DGII sowie des Bundesverbands der Augenärtzte BVA-Umfrage 1999”; results of the current survey expected in December 2008.) Approximately 1.5 million cataract operations a year are performed in the USA (Schein et al. N Engl J Med 2000). Approximately 10 million cataract operations a year are performed worldwide.

The most common complication after cataract surgery is what is known as posterior capsule opacification or aftercataract, which leads to a further reduction in central visual acuity. Aftercataract occurs in 11.8% of patients within a year of the operation to remove the lens and implant an IOL, and occurs within 5 years of operation in as many as 28.4% of patients. The highest incidence, at more than 90%, is observed in children after operation on a congenital cataract (Kugelberg et al., J Cat Refract Surg 2005, 31: 757-762).

The incidence is further dependent on the lens material and lens design; the aftercataract rate is up to 55% for PMMA lenses, but only 2.2% for hydrophobic acrylate/silicone lenses. However, the functionality of accommodative IOLs such as the 1-CU lens (Human Optics AG, Erlangen) is currently severely compromised by the extremely high 100% aftercataract rate and the subsequent need for removal (Mastropasqua et al. Acta Ophthalmol Scand 2007, 85: 409-414). Relatively high aftercataract rates of 20% have also been documented for multifocal IOLs (meta-analysis). Hydrogel lenses are also affected by relatively high aftercataract rates.

The formation of aftercataracts is caused by residual equatorial lens epithelial cells, which migrate from the equatorial region of the capsular bag which is left behind after removing the ocular lens into the centre of the optical axis, proliferate, and attach to the surface of the artificial lens, resulting in a significant reduction in central visual acuity. The surface material of the IOL appears to stimulate the lens epithelial cells, resulting in the production of cytokines (IL-1 and 6; PGE2) which interfere with the blood-aqueous barrier (mediated by PGE2) and lead to an inflammatory reaction, in addition to the aforementioned cellular reaction.

The gold standard for treating the aftercataract after formation is Nd:YAG laser capsulotomy. This ambulant procedure, in which the opacified posterior lens capsule is opened up using an Nd:YAG laser, resulting in a significant increase in visual acuity, is simple to perform. However, in the USA the annual cost of this type of treatment alone is approximately 250 million USD. Moreover, the functionality of modem IOLs such as the accommodative and multifocal lenses is severely compromised by the laser procedure; accommodative IOLs lose their capacity for accommodation (Mastropasqua et al. Acta Ophthalmol Scand 2007, 85: 409-414) and multifocal IOLs may suffer in terms of their imagining quality because of decentring of the lens in the capsular bag. In addition, there is not equal access to Nd:YAG capsulotomy in all parts of the world.

In addition, laser treatment often leads to complications, such as laser damage to the IOL (12%), a secondary pressure increase (secondary glaucoma; 8.5%) or damage to the retina (approx. 1%) as well as IOL subluxation or dislocation (0.10%).

In terms of the quality of ophthalmological care worldwide, and also for reasons of cost-effectiveness, a prophylactic measure which prevents an aftercataract from even occurring in the first place would be desirable. Thus far, according to meta-analysis, only an IOL optics design with a sharp optical edge has been found to be prophylactically effective (“sharp edge design”; Findl et al. Cochrane Database of Systematic Reviews 2007) by comparison with lenses having a round optical edge.

However, in spite of the use of IOLs having a sharp optical edge, according to current statistical analysis of data from US insurance companies and their pay-outs this lens design has not produced the cost reduction hoped for (Cleary and Spalton, ESCRS Eurotimes 2008, 115: 1308-1314). This could be due to the rather short follow-up times after cataract operation/lens implantation, which in some cases are as little as three years (Kohnen et al., Ophthalmology 2008). Moreover, it is questionable to what extent the aforementioned technical provisions as regards lens material and design are still technically practicable, to reduce the incidence of aftercataract, when designing new-generation IOLs, i.e. accommodative and multifocal IOLs.

Modifications to surgical technology and the intraoperative or perioperative application of a drug for pharmacological aftercataract prophylaxis have thus far been found not to be significant in relation to aftercataract development.

There are numerous pharmacological approaches to aftercataract prophylaxis but thus far they have not been successful in a clinical context. The following pharmacological substances have been tested thus far: mitomycin C (MMC; Chung et al., J Cataract Refract Surg 2000), ethylenediaminetetraacetic acid (EDTA; Nishi et al. J Cataract Refract Surg 1999), 5-fluoruracil (5-FU; Ruiz at al. Ophthalmic Res 1990) and daunomycin (Power et al. J Cataract Refract Surg 1994) as well as a large number of others, such as cytostatics, including antimetabolites such as methotrexate or 5-FU, antibiotics such as daunomycin or mitomycin, antimitotics such as colchicine; NSAR's such as indomethacin or diclofenac, and others such as heparin, sumarin or transferrin. Because of the ineffectiveness and toxic side effects, these substances are of limited clinical use (pilot studies). Effective substances such as mitomycin C (Kim et al. Clin Exp Opthalmol 2007), thapsigargins/5-fluorouracil (Abdelwahab et al. Eye 2008) and Triton X-100/distilled water (Maloof at al. Arch Ophthalmol 2005) necessitate the use of an irrigation system in the sealed capsule (“sealed-capsule irrigation device”) during application in the eye to inhibit the significant toxic side-effects of the substances. This results in a considerable increase in the complexity of the operation and in increased operating times. However, distilled water alone (used in the aforementioned “sealed-capsule device”) has been found to be ineffective, as could be seen in 17 patients after two years of follow-up (Rabsilber at al., Br J Ophthalmol 2007). Substances such as heparin on surface-modified hydrophilic acrylic IOLs (BioVue®, OH, Ontario, Calif., USA) are ineffective as regards the prophylactic effect thereof on the occurrence of aftercataract by comparison with hydrophobic acrylic IOLs (Sensar®, AR 40e, AMO, Santa Ana, Calif., USA), as has been shown in a current randomised clinical study on one hundred patients (Kang et al., Eur J Ophthalmol 2008). In addition, intraocular application of heparin is associated with a risk of bleeding because of the blood-thinning properties thereof.

The substances used for aftercataract prophylaxis thus far are often antitumour agents having unpleasant and undesirable side-effects, which have been documented well and characterised well pharmaceutically. In addition, the substances used thus far are mostly extremely water-soluble, and because of their high water-solubility do not adhere either to hydrophilic or to hydrophobic materials.

It was therefore an object of the present invention to provide medical materials having a favourable effect on the wound healing process.

This object is achieved according to the invention by a medical material which is characterised in that it comprises a phosphocholine compound of formula (I)

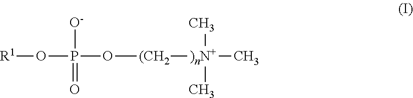

wherein
R¹ is a hydrocarbon radical optionally comprising heteroatoms and
n is an integer from 1 to 5.

It has been found that the medical material according to the invention comprising a phosphocholine compound leads to an improvement in the wound healing reaction and a reduction in foreign-body reactions because of the surface properties and biocompatibility thereof. In this context, the medical material is preferably coated with the phosphocholine compound. However, it is also possible to mix the phosphocholine compound into the base material of the medical material.

In a preferred embodiment, the phosphocholine compound is applied by immersing the medical material into a solution of the phosphocholine compound.

In accordance with the invention, it has surprisingly been found that medical materials can be coated with phosphocholine compounds. In this context, the coating may be applied directly in a simple manner, without additives, linkers, other connecting substances or precoatings being required.

The medical material according to the invention is in particular an inert biocompatible material, preferably an inert biocompatible solid material. Preferred medical materials are for example implants or medical devices.

The medical material according to the invention is in particular a polymeric material from which medical devices or implants can subsequently be formed. Adapted polymeric materials are for example hydrophilic or hydrophobic polymers, hydrophilic polymers being preferred. Examples of polymers of this type include inter alia acrylates, in particular hydrophobic acrylate or hydrophilic acrylate, methacrylates, in particular polymethyl methacrylate (PMMA) or silicones.

However, the material may also be a metal or a metal alloy, comprising for example titanium, iron and/or cobalt.

The medical material according to the invention may in particular be used for the manufacture of medical devices or implants. The invention therefore also comprises medical implants or medical material materials, used in the diagnosis, treatment, prophylaxis and/or improvement of undesirable conditions, which are formed at least in part from the medical material according to the invention. Preferably at least the part coming into contact with the body or bodily fluids is formed from the medical material. However, it is also possible to produce an entire medical implant or material from the medical material according to the invention.

Examples of materials which may be formed at least in part from the medical material according to the invention include stents, heart valves, permanent catheters, prostheses, implants and/or suturing materials and/or contact lenses.

The interface-active properties of the phosphocholines and the tendency thereof to form monomolecular films at interfaces mean that a wide range of materials can be coated with phosphocholines.

It was in particular an object of the present invention to provide ophthalmological implants which prevent or at least reduce aftercataract formation.

This object is achieved according to the invention by an ophthalmological implant which is characterised in that it comprises a phosphocholine compound and in particular an alkylphosphocholine.

It has been found that the ophthalmological implants according to the invention offer an effective, non-toxic option for pharmacological aftercataract prophylaxis. By contrast with many current approaches, according to the invention the active ingredient is a component of the ophthalmological implant, rather than merely being administered separately, for example by topical application. The active ingredient can thus be incorporated into the implant material. However, it is often particularly advantageous to provide implants with a surface coating of phosphocholines, in particular alkylphosphocholines. It has been found that the surfaces of ophthalmological implants, in particular of intraocular lenses, can be modified with phosphocholines, in particular alkylphosphocholines. This was surprising, since with many substances coating ophthalmological implants, in particular intraocular lenses, is not possible.

It has how been possible to demonstrate that with phosphocholine compounds, in particular alkylphosphocholines, ophthalmological implants and in particular intraocular lenses can be coated. This considerably facilitates application. In particular, proceeding in this manner does not result in longer operating times due to intraocular application of an active substance. Rather, the implant, in particular an intraocular lens, which has already been surface modified with the phosphocholine compounds, in particular alkylphosphocholines (APCs), can still be implanted directly as in other cases without additional operative measures.

Surprisingly, it has now been found that with ophthalmological implants which comprise phosphocholine compounds, in particular alkylphosphocholines, in particular with intraocular lenses coated with phosphocholine compounds, in particular alkylphosphocholines, occurrence of aftercataracts can be successfully reduced. This is in contrast with other proliferation-inhibiting active ingredients previously tested in vitro, which do not exhibit any prophylactic effect as a coating on intraocular lenses.

It has further been found according to the invention that phosphocholine compounds, in particular APCs, are also well tolerated in the posterior portion of the eye and in particular do not induce retinal toxicity. This is important because the operative removal of an ocular lens can also lead to a tear in the posterior lens capsule, and this can result in a connection between the anterior and posterior portion of the eye (in that the diffusion barrier is broken).

In a particularly preferred embodiment, the ophthalmological implant according to the invention is an intraocular lens. However, other implants may also be involved, for example a refractive, deformable polymer such as is used in refractive lens surgery known as “lens refilling” (Nishi et al., J. Cat. Refract. Surg. 1998 and 2008; Koopmans et al., Invest Ophthalmol V is Sci 2003 and 2006). In this field of application, the polymer is preferably mixed with phosphocholine compounds, in particular alkylphosphocholine.

Incorporating or coating an ophthalmological implant with phosphocholine compounds, in particular an alkylphosphocholine, modifies the surface of the implant.

According to the invention the surface of an ophthalmological implant, in particular the surface of intraocular lenses, is particularly preferably coated with an alkylphosphocholine, in particular oleylphosphocholine, erucyiphosphocholine (ErPC) or erucylphosphohomocholine (erufosin; ErPC₃), in particular so as to form a monomolecular surface film.

The phosphocholine compounds which can be used according to the invention are in particular lipophilic phosphocholine compounds. Lipophilic phosphocholine compounds of this type preferably have at least one hydrocarbon radical, in particular a saturated alkyl radical or a hydrocarbon radical comprising one or more unsaturated double bonds, containing at least 12 C atoms, more preferably at least 14 C atoms. Using lipophilic phosphocholine compounds, in particular lipophilic alkylphosphocholines, makes simple coating of medical materials, in particular ophthalmological implants, possible.

Preferably, a phosphocholine compound is used which is interface-active and in particular has a pharmaceutical effect and particularly preferably has an antiproliferative, antiparasitic, antimycotic and/or antibacterial effect. Preferably, a phosphocholine compounds having an antiproliferative effect is used.

It is preferred according to the invention for the phosphocholine compound to be of formula (I)

R¹—)—PO₂⁻—O—(CH₂—)ₙ—N⁺(CH₃)₃

wherein R¹ is a hydrocarbon radical optionally comprising heteroatoms and n is an integer from 1 to 5. n is particularly preferably 2 or 3. R¹ is in particular a C₁₆-C₃₀, more preferably C₁₆-C₂₄ hydrocarbon radical. R′ may be a saturated alkyl radical, but it may also comprise one, two, three or more unsaturated bonds, in particular cis double bonds. Hydrocarbon radicals which comprise at least one cis double bond are particularly preferred, the radical R¹ particularly preferably being an erucyl (C_{22:1-cis:ω-9 radical}) or oleyl (C_{18:1-cis-ω-9 radical}) radical. In a preferred embodiment, R¹ is a hydrocarbon radical which does not comprise any heteroatoms.

It is further preferable for the radical R¹ to be a radical R²=—CH₂—(CH₂)ₓ—CH₂—O—R⁴ or a radical R³=—CH₂—CH[—O—(CH₂)_{y}—H]—CH₂—O—R⁴, wherein

R⁴ is a hydrocarbon radical, in particular a C₁₆-C₂₄ hydrocarbon radical,
x represents an integer from 0 to 4 and
y represents an integer from 1 to 3.

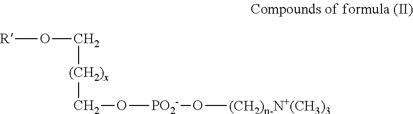

wherein

### R′=R¹;

n=an integer from 1 to 5;
x=0 to 4;
and of formula (III)

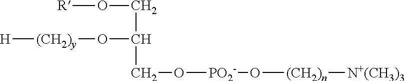

wherein

### R′=R¹;

n=an integer from 1 to 5;
y=1 to 3
are particularly preferred.

In accordance with the invention the phosphocholine compound is most preferably an alklyphosphocholine of formula (I)

R¹—O—PO₂⁻—O—(CH₂—)ₙ—N⁺(CH₃)₃

wherein R¹ is a hydrocarbon and n is an integer from 1 to 5. n is particularly preferably 2 or 3.

R¹ is in particular a C₁₆-C₃₀, more preferably a C₁₈-C₂₄ hydrocarbon radical. This radical may be an alkyl radical, but it may also comprise one or more unsaturated bonds. Hydrocarbon radicals which comprise at least one cis double bond are particularly preferred. The radical R¹ is particularly preferably an erucyl (C₂₂) or oleyl (C₁₈) radical.

The phosphocholine compound is preferably an alkylphosphocholine, an (ether) lysolecithin or an analogous substance as disclosed for example in EP 1 827 379. It is preferably not a lecithin.

Most preferably, the phosphocholine compound is erucylphosphocholine, erucylphosphohomocholine or oleylphosphocholine.

The substances, having an alkyl chain length of 16 to 24 carbon atoms, which are preferably used according to the invention are not dissolved or monodispersely distributed in water. Rather, they form superlattices in water and easily intercalate into interfaces or membranes. Phosphocholine compounds thus form a coating on materials, in particular on medical materials such as medical implants, preferably on ophthalmological implants and in particular on intraocular lenses. Further, phosphocholines intercalate easily into polymers having hydrophobic interfaces or surfaces.

Medical materials, in particular ophthalmological implants, are preferably coated with a phosphocholine compound in such a way as to lead to the formation of a monomolecular surface film.

Phosphocholine compounds and in particular alkylphosphocholines (APCs) are effective inhibitors of ocular cell proliferation, migration and adhesion in non-toxic concentrations. As synthetic phospholipid derivatives, they represent a new class of pharmacologically active substances (Eibl H et al., Cancer Treat Rev 1990) and are successful in clinical use because of the good antitumour (Leonard et al., J Clin Oncol 2001; Miltex®, Zentaris GmbH, Frankfurt) and antiparasitic (Sundar et al. N Engl J Med 2002; Impavido®, Zentaris GmbH, Frankfurt) properties thereof.

Further in vivo studies on the eye in appropriate animal test subjects have demonstrated high effectiveness and tolerance with local administration by intravitreal application (injection into the vitreous body, i.e. into the posterior portion of the eye). Thus, in rat eyes no damage to the retinal function could be detected either morphologically, by light and electron microscopy, or functionally, by taking an electroretinogram (ERG), seven days after a single injection of erucylphosphocholine (ErPC) into the vitreous body (Schüttauf et al., Curr Eye Res 2005). In rabbit eyes, it could be demonstrated that a single intravitreal administration of liposomal ErPC and free ErPC₃, one day after experimentally induced retinal detachment, achieves a significant inhibition of the intraretinal proliferation of Müller glial cells and subretinal pigment epithelial cells, without morphologically detectable toxic side-effects (Eibl et al., IOVS 2007 and Der Ophthalmologe 2007). Long-term analyses of the tolerance of ErPC in cat eyes have also produced no sign of retinal toxicity 28 days after intravitreal administration. Functional analyses on a retina ex vivo test subject at a concentration of up to 25 μM in the total solution did not produce any changes in the electroretinogram of rat retina incubated with ErPC₃ (see also example 5 of the present document). Comparable analyses on this test subject demonstrate retinal toxicity for other intraocular substances, such as triamcinolone, at the clinically administered doses (4 mg/ml) (Lüke et al., Exp Eye Res 2008).

According to the invention, it has now been found that in addition to the high effectiveness and the lack of toxic side-effects, phosphocholine compounds, in particular APCs, are adapted for incorporation into ophthalmological implants or for coating ophthalmological implants therewith.

The possibility of coating an intraocular lens with phosphocholine compounds, in particular APCs, is the ideal technical implementation of the aforementioned findings in the clinical context and is therefore of clinical relevance.

A further possibility for application is in the field of what is known as “lens refilling”. In this context, the ocular lens is operatively removed and the capsular bag is filled with a polymer of a different composition. This polymer acts as a resilient, potentially deformable substitute for the opacified, “inflexible” lens of the older patient, which has lost its deformability (capacity for accommodation) and thus its ability to read. In many patients, this presbyopia starts as early as age 45, and they therefore turn to reading glasses. With refractive lens exchange and implantation of an accommodative lens or multifocal lens, or with the new approach of “lens filling”, these patients can do without reading glasses, which are found by many to be a nuisance. The injected polymer has similar optical properties to the natural deformable lens of a young person. However, the main difficulty is the post-operative formation of an aftercataract, which worsens the acuity of vision again. Because of the chemical structure thereof, phosphocholine compounds, in particular APCs, offer the possibility of being mixed with the polymer so as to be introduced into and remain in the eye as a component of the new lens.

In addition, phosphocholine compounds, in particular APCs, can be used intraoperatively for rinsing the capsular bag before injecting the polymer or for post-operative aftercare.

In addition, phosphocholine compounds, in particular APCs, may also be applied intraocularly post-operatively, for example into the anterior and/or posterior portion of the eye, by intraocular injection, so as to provide therapeutic treatment, in addition to the prophylactic application as disclosed herein, if an aftercataract forms post-operatively, should the need for aftercare arise post-operatively.

Ophthalmological implants generally consist of materials such as acrylate materials, silicone materials or hydrogels, in particular hydrophobic acrylates, hydrophilic acrylates, silicone and preferably PMMA.

For example, hydrophobic intraocular lenses, which may be formed from acrylate or silicone, or hydrophilic intraocular lenses, which are formed from acrylate, are preferred according to the invention.

It is extremely difficult to form a coating of materials of this type. One reason for this is the high water-solubility of the active ingredients used, such as 5-fluorouracil, methotrexate, Triton X-100. In particular, the active ingredients are often detached as soon as the intraocular lenses come into contact with an aqueous medium as is present in the eye.

It has now been found that implant materials of this type can be coated outstandingly well with phosphocholine compounds, in particular alkylphosphocholines, and that phosphocholine compounds, in particular alkylphosphocholines, can readily be incorporated into implant materials.

Lipophilic phosphocholine compounds in particular are outstandingly well adapted for coating implant materials. In this context, the phosphocholine compounds may be applied to the implant materials directly as a coating, without additives, without linkers and without a precoating or other measures being required.

Phosphocholine compounds, and in particular alkylphosphocholines, as disclosed herein, have special properties and form superlattices, known as micelles, in water, which can be characterised well by way of the critical micelle concentration. These micelles intercalate spontaneously into lipophilic surfaces and interfaces and cannot be removed again, or can only be removed again with great difficulty, by aqueous media, unlike water-soluble substances, which can easily be detached.

According to the invention, it has been found that medical materials, and in particular ophthalmological implants, can be coated with phosphocholine compounds, and in particular with alkylphosphocholines, in a simple manner.

It has in particular been found that for coating intraocular implants with APCs, no additional additives, linkers or other modifications to the surfaces or of the material, for example of lenses, is required. Rather, the phosphocholine compounds, in particular APCs, can be applied directly to the surface of the medical material as a coating, or incorporated into the medical material, directly, for example directly from the solution, for example from an aqueous solution, for example from a physiological saline solution or from water, or an alcoholic, in particular ethanolic or methanolic solution. Even after repeated washing with aqueous solutions, for example with aqueous physiological saline solution, the phosphocholine compounds, for example APCs, continue to adhere to the surface of the ophthalmological implant.

In this context, the amount of incorporated or applied phosphocholine compound, in particular APC, is advantageously from 0.1 to 200 μM, in particular from 1 to 100 μM of phosphocholine compound, in particular APC, based on the mass of the ophthalmological implant, or 1 to 600 pmol, preferably 2 to 100 pmol and in particular 4 to 8 pmol phosphocholine compound, in particular APC, per mm² surface area of the medical implant, for example of an ophthalmological implant, in particular a monomolecular layer of phosphocholine compound, in particular ARC, on the surface.

It has further been found that ophthalmological implants, for example intraocular lenses, coated with phosphocholine compounds, and in particular with APCs, are outstandingly effective in the prophylaxis of aftercataract. In particular, the pharmacological effect of the intraocular implants does not only develop immediately after implantation, but they have a longer-term prophylactic effect, in particular on cellular proliferation, migration and attachment, i.e. on the wound healing process as a whole. This is extremely important in preventing the occurrence of aftercataract, since this process may develop slowly post-operatively over up to 5 years. In this context, the lens epithelials left in the capsular bag work their way slowly into the optical axis, and often do not lead to complaints on the part of the patient, such as clouded, dark or unfocussed vision, loss of vision or difficulties with reading, until months or years after the operation. With intraocular lenses coated according to the invention, occurrence of aftercataract can also be prevented effectively in the long term.

In summary, it can be found that phosphocholine compounds, and in particular alkylphosphocholines, are outstandingly adapted for application in pharmacological aftercataract prophylaxis because of the antiproliferative properties thereof, the high tolerance thereof and the compatibility thereof with ophthalmological implant materials. The intraocular lenses cannot be distinguished by the naked eye from uncoated control intraocular lenses of the same batch. Surface modification of intraocular lenses with phosphocholine compounds, and in particular alkylphosphocholines, is of particular interest. In addition, phosphocholine compounds, and in particular alkylphosphocholines, may be used to rinse the capsular bag before implanting a new, artificial lens and as an intraocular injection into the anterior and/or posterior portion of the eye as required. Because of the high acceptability, additional operational measures, such as the use of a “sealed capsule” system, are not necessary with phosphocholine compounds, and in particular alkylphosphocholines.

According to the invention, it has further been found that phosphocholine compounds, and in particular APCs, are outstanding for use for the treatment and/or prophylaxis of ophthalmological disorders, in particular ophthalmological disorders of which the pathogenesis includes cellular proliferation, migration, attachment and/or contraction.

The invention therefore also relates to the use of phosphocholine compounds for the preparation of a drug for the prophylaxis, prevention and/or treatment of ophthalmological disorders.

In particular alkylphosphocholines and more preferably C₁₆-C₂₄ alkylphosphocholines, as mentioned previously herein, are preferably used as phosphocholine compounds. They may for example be administered topically or systemically.

It has been found that phosphocholine compounds are outstandingly adapted in particular for treating the following ophthalmological disorders:
disorders of the eyelid or ocular adnexa, in particular infection, inflammation, post-operative conditions, scars, trauma, cosmetic indications, malignancy, proliferative disorders, dermal lesions.
disorders of the conjunctiva and/or of the ocular surface, in particular infection, inflammation, post-operative conditions, scars, traumas, cosmetic indications, malignancy, proliferative disorders, conditions induced by contact lenses, lubrication.
disorders of the cornea and/or the ocular surface, in particular infection, inflammation, post-operative conditions, scars, traumas, cosmetic indications, malignancy, proliferative disorders, conditions induced by contact lenses, lubrication, sicca syndrome.
use as a post-trabeculectomy agent, in particular as an anti-scarring agent.
for the medical treatment of glaucoma, for IOP reduction and/or for facilitating the drainage of aqueous fluid.
in cataract operations, for preventing aftercataract.
retinal disorders, proliferative vitreoretinopathy, retinal detachment, diabetic retinopathy (NPDR; PDR; maculopathy), age-related macular degeneration, macular disease (CSR; CNV).
uveitis, intraocular infection, intraocular inflammation, intraocular malignancy and/or traumas.

Phosphocholine compounds can be used in various ways in ophthalmological disorders, preferably by way of surface modification and intraocular lenses, as a coating agent and/or component in contact lenses, as an additive to polymers for intraocular use (for example lens refilling), as an additive or coating in intraocular devices, as an additive or coating in delayed-release systems, by intraocular, parabulbar, retrobulbar and/or episcleral injection, as eye drops and/or by systemic administration (for example orally, intravenously, subcutaneously, intramuscularly).

A further problem which often occurs after a cataract operation is what is known as “lens glistening”, i.e. glinting effects on the lens. Interfering glistening effects occur in approximately 40 to 67% of all patients who have a hydrophobic, pliable acrylic lens implanted in a cataract operation. It has now been found that by coating lenses of this type with alkylphosphocholines, the formation of these glistening effects or flecks can be prevented or at least reduced. In this way, the biocompatibility of lenses of this type can be improved. A further subject-matter of the invention is therefore the use of phosphocholine compounds to reduce glistening effects in intraocular lenses. It is assumed that alkylphosphocholines can reduce glistening effects which result from contact between the hydrophobic lens surface and the hydrophilic aqueous medium.

The invention is explained further by way of the appended drawings and the following examples.

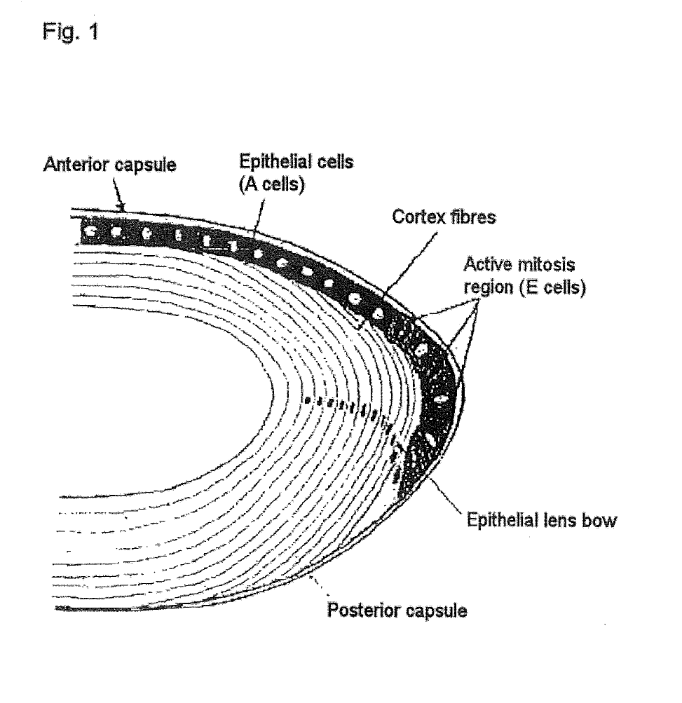

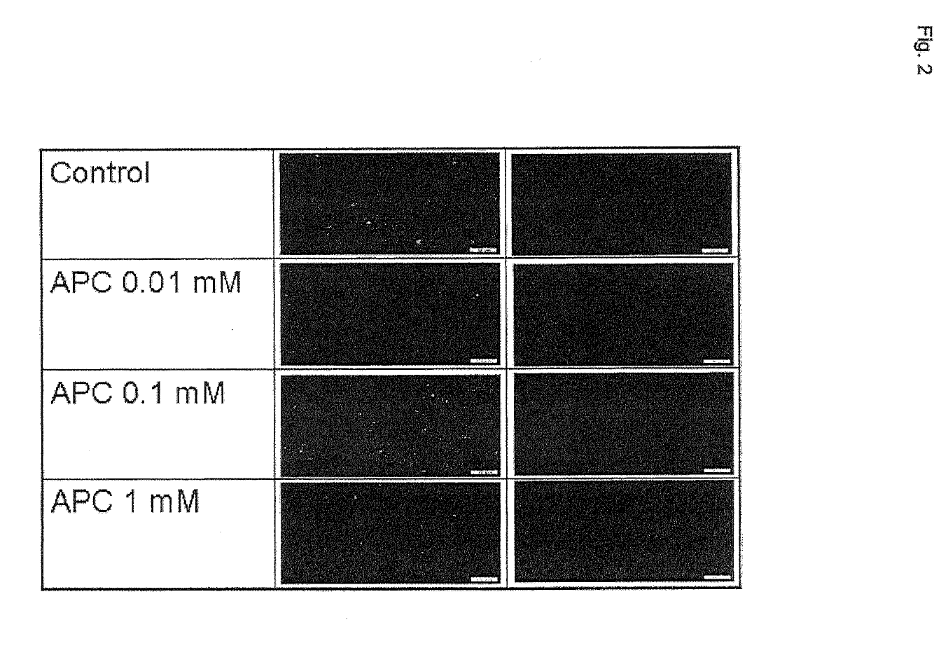

FIG. 1 schematically shows the pathogenesis of the aftercataract (Nishsi et al., J Cat Refract Surg 1996).

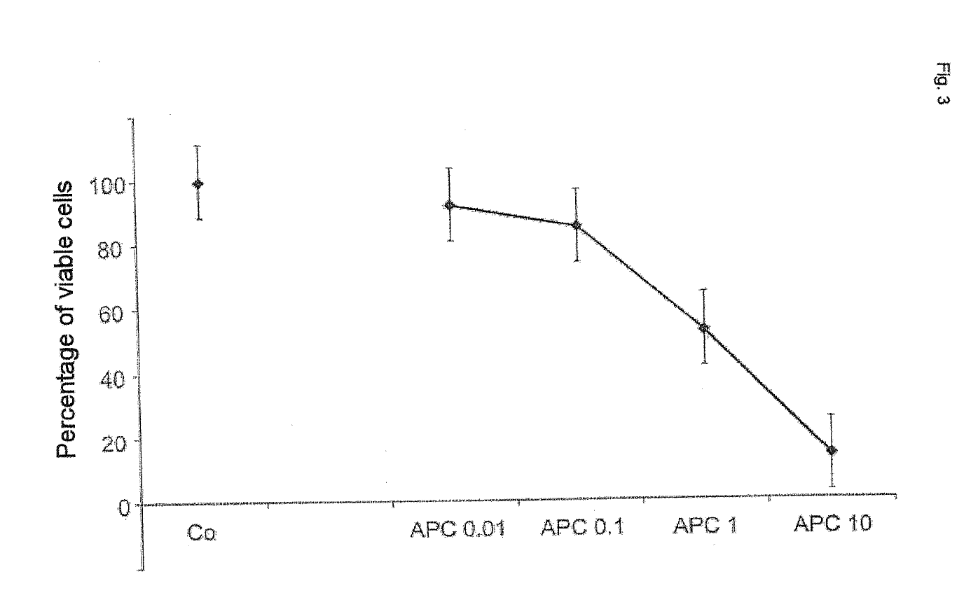

FIG. 2 shows the results of the cell viability analysis determined by the trypan blue exclusion test and the live/dead assay, in which no difference between APC-treated cells and control cells can be established.

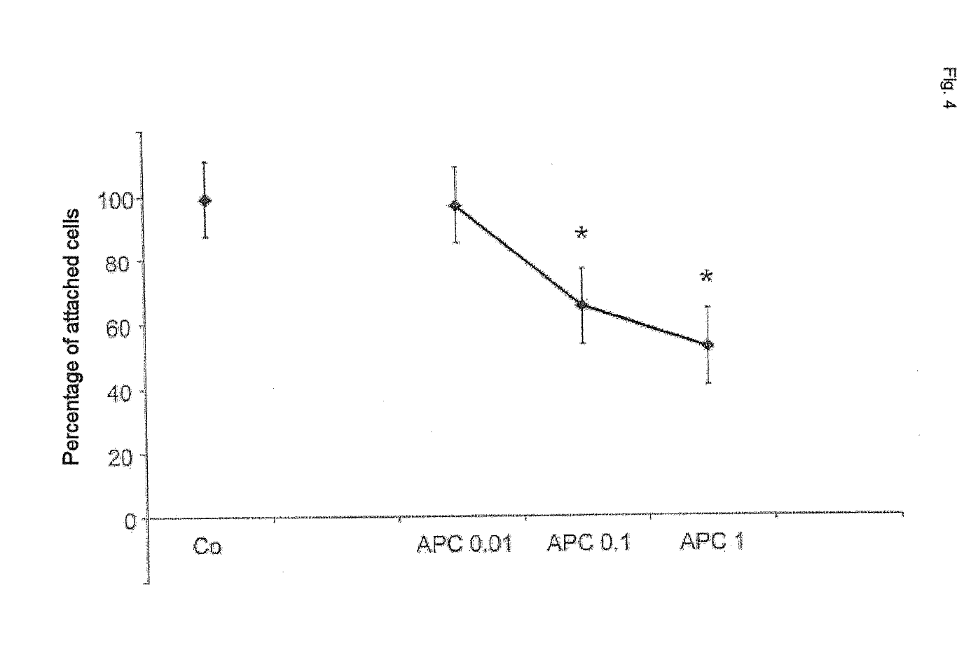

FIG. 3 shows the inhibition of human lens epithelial cell proliferation by APCs as a function of concentration.

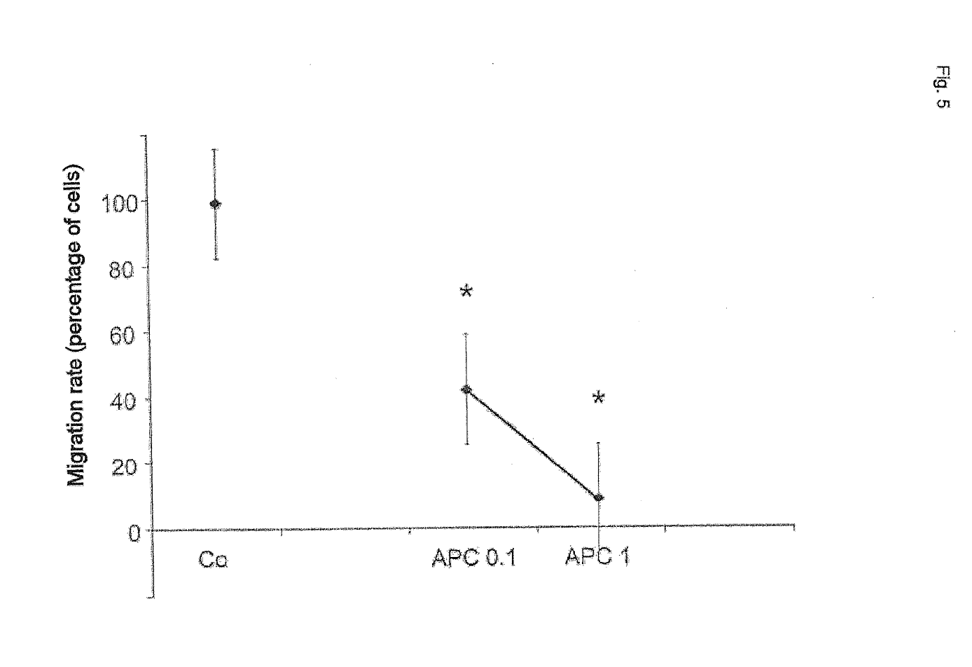

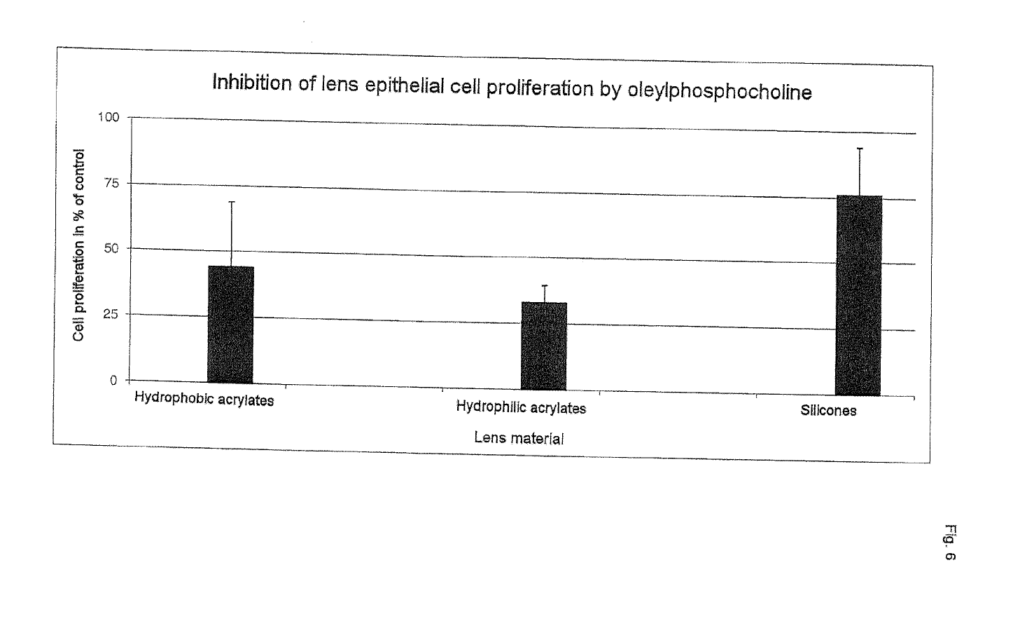

FIG. 4 shows the inhibition of cell attachment of human lens epithelial cells by APCs as a function of dosage.

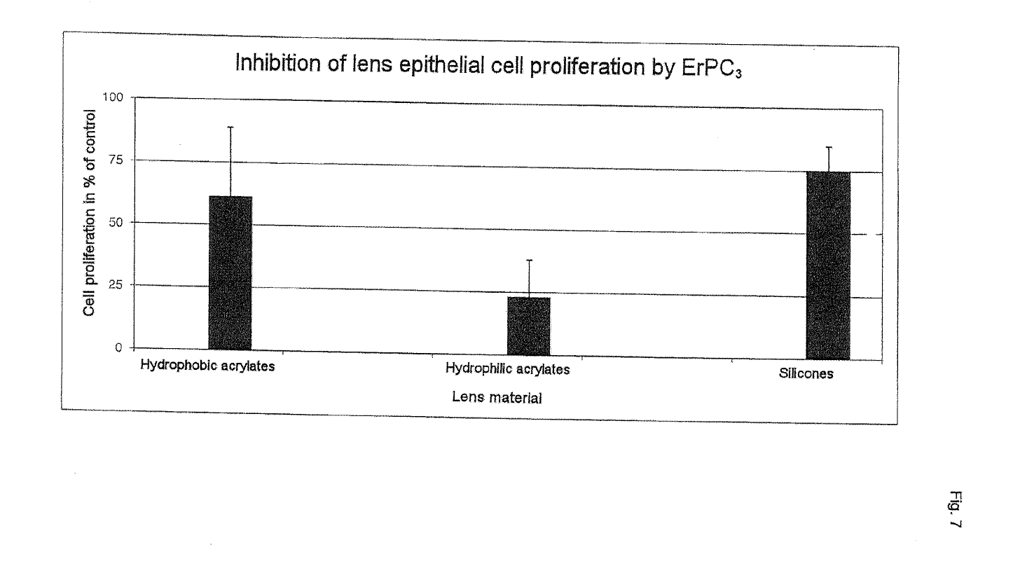

FIG. 5 shows the inhibition of the migration of human lens epithelial cells as a function of dosage.

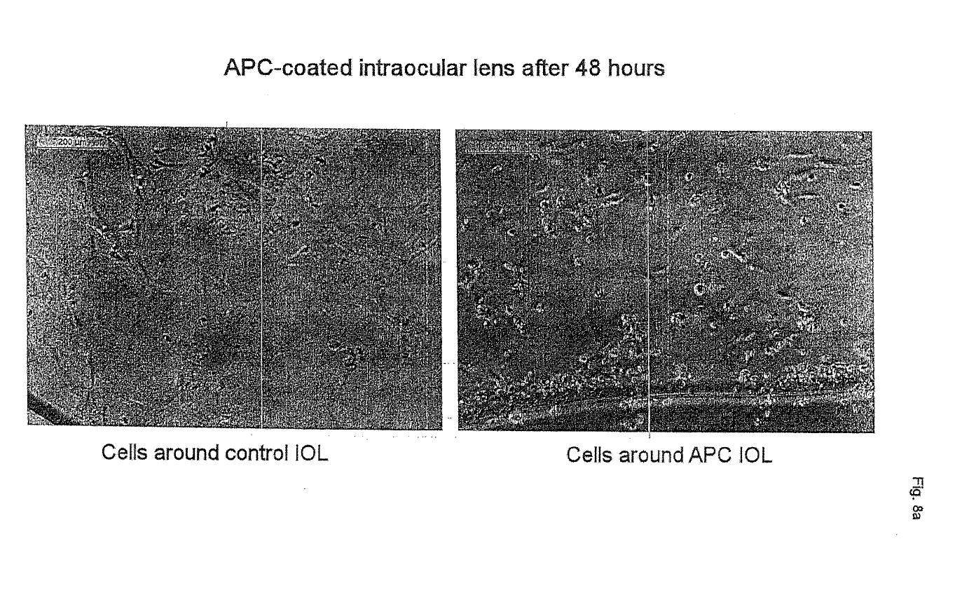

FIG. 6 shows the inhibition of human lens epithelial cell proliferation by oleylphosphocholine-coated intraocular lenses in the form of a bar chart.

This shows that intraocular lenses made of various materials (hydrophobic acrylates, hydrophilic acrylates and silicones) and having various haptic designs and optical diameters, coated with alkylphosphocholines (OIPC), can inhibit the cell growth of lens epithelial cells which are already proliferating.

A tetrazolium reduction assay [MTT (3-[4,5-dimethylthiazol-2-yl]-2,5-diphenyltetrazolium bromide] (Sigma-Aldrich) was carried out to determine the cell count. The intraocular lenses were removed, the cells were washed with PBS and incubated with the MTT reagent for 1 hour at 37° C. Only vital cells metabolise the solution. Subsequently, the cell membrane was dissolved with DMSO (dimethylsulphoxide) and the optical thickness was measured in the ELISA reader at 550 nm. In this case, the optical thickness correlates with the number of proliferating cells.

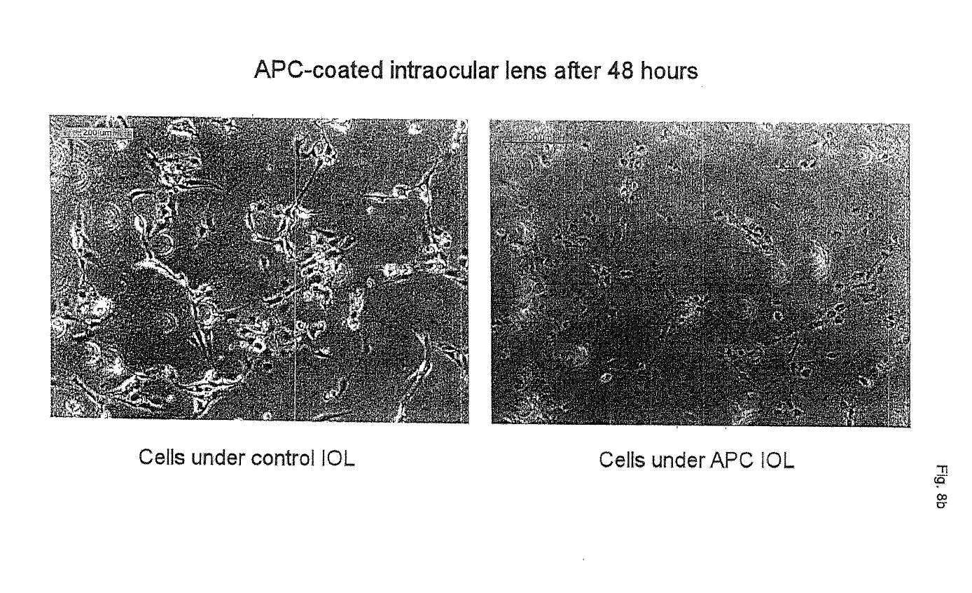

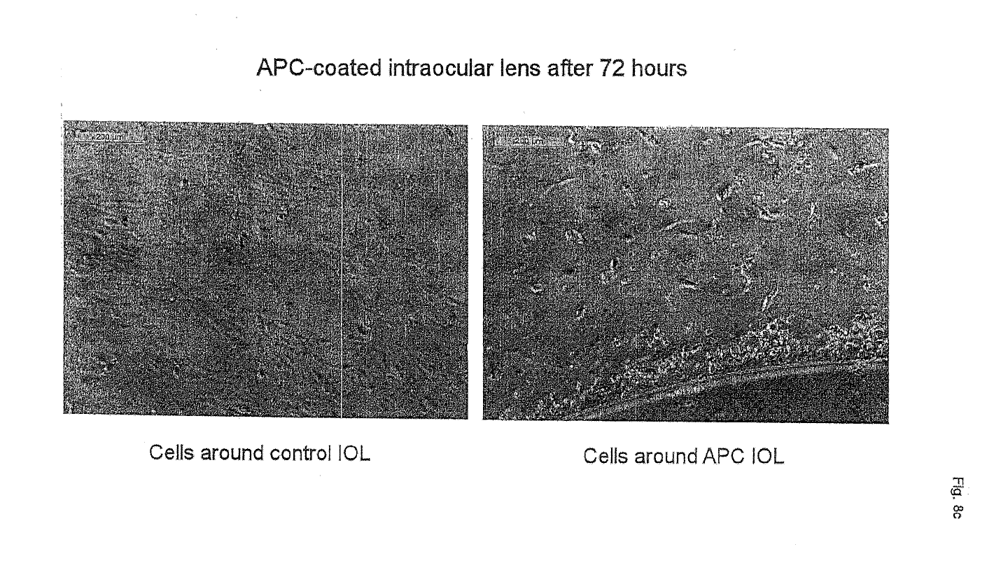

FIG. 7 shows the inhibition of human lens epithelial cell proliferation by erucylphosphohomocholine (ErPC3)-coated intraocular lenses in the form of a bar chart.

This shows that intraocular lenses made of various materials (hydrophobic acrylates, hydrophilic acrylates and silicones) and having various haptic designs and optical diameters, coated with alkylphosphocholines (ErPC₃), can inhibit the cell growth of lens epithelial cells which are already proliferating. This was determined as described for FIG. 6.

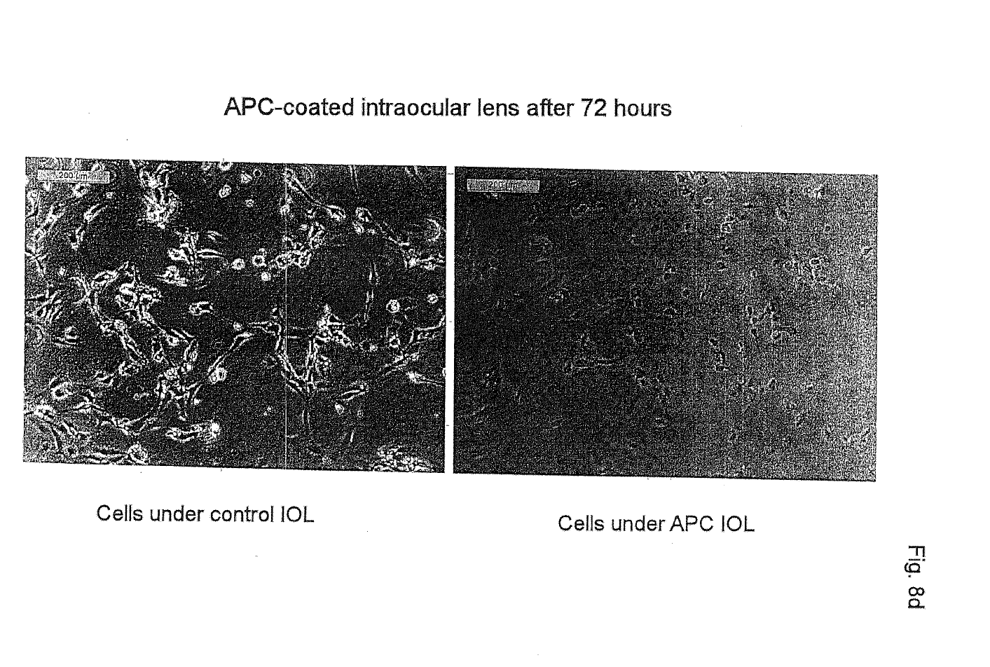

FIG. 8 shows human lens epithelial cells in cell culture. The readings were taken at 100× magnification in a phase contrast microscope.

FIG. 8a shows lens epithelial cells around an uncoated control IOL (left) and around an APC-coated IOL (right) after 48 hours.

FIG. 8b shows lens epithelial cells around an uncoated control IOL (left) and around an APC-coated IOL (right) after 48 hours.

FIG. 8c and FIG. 8d show the effects after 72 hours in each case.

When introduced into a culture, an alkylphosphocholine-coated intraocular lens leads to inhibition of the cell propagation and the growth of the cells, both around and below the intraocular lens. The effect strengthens over time (increase in the effect from 48 to 72 hours after introducing the alkylphosphocholine-coated intraocular lenses into the cell culture).

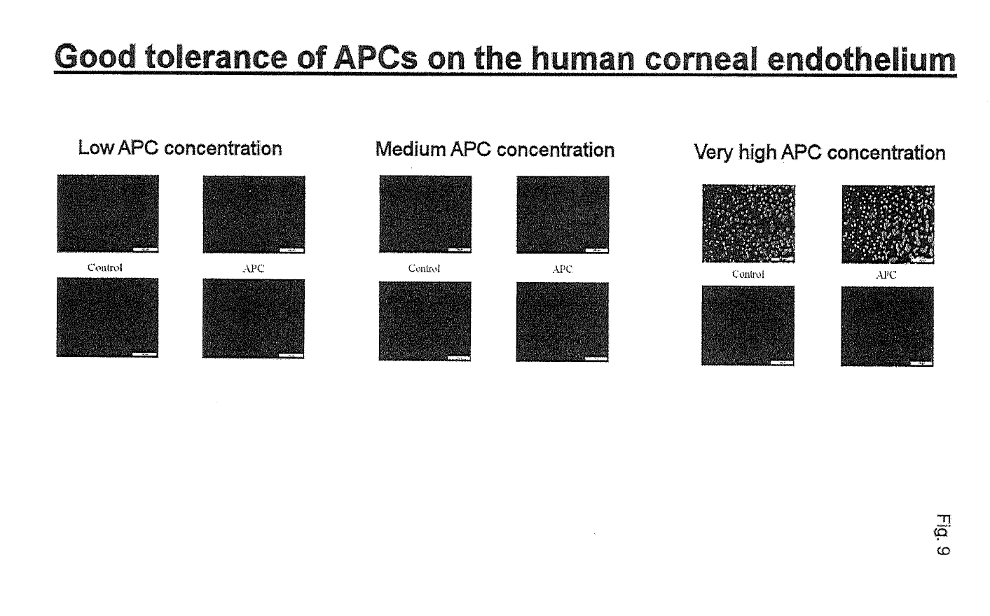

FIG. 9 shows the biocompatibility of the alkylphosphocholines on the human corneal endothelium on the anterior portion of the eye.

After carrying out live/dead colouring, no dead endothelium cells can be seen at alkylphosphocholine concentrations of up to 1 mM (red colouring of the cells by propidium iodide).

A live/dead test was carried out. In this case, cell nuclei of non-vital cells were coloured red, since propidium iodide can only penetrate into dead cells. Cell nuclei of vital cells were coloured blue with the membrane-permeable dye Hoechst 33342 (Intergen). Beforehand, the cells had been seeded on four-compartment substrates and incubated at APC concentrations of 100 μM (low APC concentration), 1 mM (medium ARC concentration) and 10 mM (very high APC concentration) for 24 hours at 37° C. under standard cell culture conditions. The ratio of vital to non-vital cells was determined by counting under the epifluorescence microscope (Leica DMR).

### EXAMPLES

### Example 1

### Monomolecular Coating of an Intraocular Lens with an Alkylphosphocholine

1.1 Theoretical calculations

The following applies when calculating the likelihood of “modification of the surface of intraocular lenses with alkylphosphocholines, in particular erucylphosphocholine (ErPC) and erucylphosphohomocholine (erufosin; ErPC₃), assuming formation of a monomolecular surface film”.

The total surface area of the intraocular lens (d=4 mm; r=2 mm) is based on a spherical surface (S):

$$\begin{matrix}s = 4 * π * {r}^{2} \\ = 4 * 3.14 * 2 \\ = 50 {mm}^{2} \\ = 50 * {10}^{- 6} {m}^{2} ( total surface area of the \vert OL ) .\end{matrix}$$

Taking the specific surface load of the alkylphosphocholines on the water-air interface (Langmuir-Trog) gives a value of approximately 25 Å²/molecule=25*10⁻²⁰ [m²].

Dividing the surface area of the lens by the specific surface load of an individual molecule gives the number of molecules (Z) which can intercalate into the lens interface:

Z=50*10-6 [m2] 25*10-20 [m2]

Z=2*10¹⁴

For erufosin, with MW 503.75:

1 mol=504 g=6*10²³ molecules

There are 2*10¹⁴ molecules on a lens. Therefore, for erufosin:

1 molecule=504 [g] 6*10-23=84*10-23 g

The weight of a single molecule of erufosin is therefore 84*10⁻²³ g.

On the lens there are:

$$\begin{matrix}2 * {10}^{14} molecules = 2 * {10}^{14} * 84 * {10}^{- 23} \\ = 1680 * {10}^{- 10} [ g ] ( \begin{matrix}from MW = 504 ; \\ conversion from g to mol\end{matrix} ) \\ \hat{=} 3 * 10 [ mol ] \\ = 0.3 [ nmol ] = 300 [ pmol ]\end{matrix}$$

These considerations show that the ErPC₃ molecules intercalated into a lens surface are sufficient for determining the amount of ErPC₃ quantitatively by mass spectroscopy.

1.2 Experimental coating of intraocular lenses

10 intraocular lenses were each incubated separately in a test tube with 200 μl highly concentrated erufosin parent solution (10 mM ErPC₃ in methanol or water). The lens was subsequently transferred into a new test tube and dried. To extract the erufosin adhering to the lens, the lens was rinsed with methanol/acetonitrile (in the ratio 9:1). Erufosin is highly soluble in this solvent mixture, and erufosin molecules adhering to the lens are removed in this extraction step. The amount of erufosin is subsequently quantitatively determined by mass spectrometry. In this context, an amount of ErPC of between 280 and 330 μmol per lens is measured. The experimentally determined amount corresponds directly to the theoretically calculated amount for a coating consisting of a monomolecular film of erufosin.

Other medical materials may also be coated with phosphocholines in the same way.

1.3 Stability of the coating 10 intraocular lenses were coated with erufosin, as described in example 1.2. The lenses were subsequently each separately transferred into new test tubes and subjected to four washing steps with physiological saline solution. In a further extraction step using methanol/acetonitrile (ratio 9:1), the erufosin adhering to the lenses was removed and determined by mass spectroscopy. The amount of erufosin determined was between 280 and 330 μmol per lens. Even with repeated washing or rinsing with physiological saline solution, no noticeable removal of the erufosin coating from the lenses was established.

### Example 2

### Effect of Alkylphosphocholines on Human Lens Epithelial Cells

2. Cell culture of human lens epithelial cells

The human lens epithelial cell line HLE-B3 was cultivated in Eagle's modified essential medium (MEM; Biochrom, Berlin, Germany), supplemented with FCS, 50 IU penicillin/ml and 50 μg streptomycin/ml at 37° C. in an incubator in a 5% carbon dioxide atmosphere. The medium was changed every three days. Trypsin EDTA was used to subcultivate cells according to confluence after 5 to 7 days. The cellular growth was observed daily using a Leica phase contrast microscope.

To determine the proliferation characteristics, the growth was measured at various points in time by cell counting using a Neubauer chamber and an automated cell counter (CASY 1, Innovation, Karlsruhe, Germany). The maximum proliferation was measured after 72 hours.

### 2.2 Alkylphosphocholines

The alkylphosphocholines erucylphosphocholine and erucylhomophosphocholine (erucyl-(N,N,N-trimethyl)-propylammonium) were used. The alkylphosphocholines were dissolved in PBS and stored at 4° C. Using a dilution series in PBS, final APC concentrations in equal volumes of PBS were obtained. As a control, the same volumes of PBS without addition of APCs were used in all of the experiments.

2.3 Cell viability assay

The cell viability of HLE-B3 cells was evaluated by two different methods, the trypan blue exclusion test and the live/dead assay. The trypan blue exclusion test was carried out as follows. After the incubation period, samples of HLE-B3 cells were counted in a haemocytometer chamber by the trypan blue exclusion method and the fraction of dead cells was calculated.

The live/dead assay, a two-colour fluorescence assay, was used to quantify the cell viability. Nuclei of non-viable cells appeared red because they had been coloured with the membrane-impermeable dye propidium iodide (Sigma Aldrich), whilst the nuclei of all of the cells had been coloured with the membrane-permeable dye Hoechst 33342 (Intergen, Purchase, N.Y.). Virtually confluent cultures of HLE-B3 cells, grown on four chambers, were treated with three different concentrations of APCs for 24 hours. To evaluate the cell viability, the cells were washed with PBS and incubated with 2.0 μg/ml propidium iodide and 1.0 μg/ml Hoechst 33342 for 20 minutes at 37° C. Subsequently, the cells were analysed with an epifluorescence microscope (Leica DMR, Bensheim, Germany). Representative areas were then digitally photodocumented (Leica Image Capturer, Bensheim, Germany). The marked nuclei were counted in fluorescence photomicrographs. Dead cells were given as a percentage of the total nuclei in the field. The data are based on counts in three experiments, each carried out in duplicate depressions, with four documented representative fields per depression.

The APC concentrations used were 0.01 mM, 0.1 mM and 1 mM. This interval was selected so as to cover the estimated IC₅₀ concentration and to determine in vitro the cell viability at potential effective concentrations for the inhibition of proliferation, attachment and migration. No morphological cell changes could be observed in the phase contrast microscope for any of the analysed concentrations. The cytotoxicity as determined by the trypan blue exclusion test and the live/dead assay was no different for APC treated cells and the control cells (see FIG. 2).

2.4 Cell proliferation assay

The tetrazolium dye reduction assay (MTT, 3-[4,5 dimethyl thiazol-2-yl]-2,5-diphenyltetrazolium bromide) was used to determine the survival of the cells. HLE-B3 cells (150 μl/depression at a density of 5×10⁴ cells/depression) were placed in 96-well plates and treated with various concentrations of APCs over 24 hours. The various concentrations of 0.01 mM, 0.1 mM and 1 mM covered the 50% inhibition concentration (IC₅₀) as determined by preliminary assays. The IC₅₀ is defined as the concentration of an active ingredient which leads to a 50% reduction in the cell count at non-toxic concentrations.

The MTT test was carried out as described in Mosmann, with some modifications. After removing the medium, the cells were washed with PBS and the MTT solution was added. The cells were incubated at 37° C. for 30 minutes. After three washing steps with PBS (pH 7.4), the insoluble formazan crystals were dissolved in dimethyl sulphoxide. The optical density in the depressions was determined using a microplate readout device at 550 nm (molecular probes, Garching, Germany). The results from the “wells” were given as the average percentage of the control proliferation. The experiments were carried out in triplicate and repeated three times. HLE-B3 cells of the same passage which were only incubated with PBS were used as a control.

The intraocular lenses coated with alkylphosphocholines are introduced into a cell culture volume of 500 μl, which corresponds to the volume of the human lens capsular bag in which the intraocular lenses are implanted in the human cataract operation. Moreover, the coated intraocular lenses in vitro are introduced into a cell culture with human lens epithelial cells which are already proliferating. These are more difficult conditions than are encountered in vivo, since the cells are already activated.

The HLE-B3 proliferation was reduced significantly after a single treatment of the cells with APCs, which were added to the culture medium in the presence of serum. The following concentrations were adapted for inhibiting proliferation: 0.01 mM (p=0.0048); 0.1 mM (p<0.001) and 1 mM (p<0.001) (see FIG. 3). The observed effect was concentration-dependent and the IC₅₀ concentration was determined to be approximately 0.1 mM.

2.5 Cell attachment assay 96-well plates (Nunc, Wiesbaden, Germany) were coated with 70 μl/depression fibronectin (50 pg/ml in PBS [pH 7.4]; Sigma-Aldrich) for 16 hours at 4° C. Unspecific bonding was blocked by 2 mg/ml ovalbumin (Sigma-Aldrich) in PBS for one hour at 37° C. HLE-B3 cells were placed in 96-well plates (Nunc) at a density of 1×10⁵ cells/depression in 1 ml MEM, 20% FCS. APCs were added at three different concentrations (0.01 mM, 0.1 mM and 1 mM). After four hours, the cells were carefully washed three times using an automated plate washing device (molecular devices, Garching, Germany).

With the tetrazolium dye reduction assay (MTT) (Sigma Aldrich), the number of cells attached after the washing step was determined, as described above. The number of attached viable cells correlated with the absorbance (optical density, OD), measured by the MTT test, at 550 nm. The results for the depressions were expressed as the average percentage of the control (control OD at 550 nm denoted as 100%).

After a single treatment of HLE-B3 cells, APCs were able to induce significant inhibition of cell attachment in a manner dependent on the dosage (see FIG. 4). Close to the IC₅₀ concentration, as determined previously by the MU test, APCs reduced cell adhesion very effectively to 66.5% (p<0.001 at 0.1 mM) by comparison with controls. The maximum inhibition of cell attachment was achieved at an APC concentration of 1 mM (54.1%).

2.6 Cell migration assay

Migration was determined by a modification of the Boyden chamber method, using microchemotaxis chambers (Neuroprobe, Gaithersburg, Md., USA) and polycarbonate filters (Nucleoprobe, Karlsruhe, Germany) having a pore size of 8.0 μm. A fibronectin-coated filter, comprising 180 μl MEM with epidermal growth factor (EGF-BB, PeptroTech, London) in a concentration of 20 ng/ml, was arranged on top of the lower half of the chamber. The upper half of the chamber was filled with a suspension of HLE-B3 cells at a density of 2×10⁵ cells/rill of MEM and 1% of FCS (500 μl). APCs were added at two different concentrations, namely 0.1 mM and 1 mM. The cells were incubated for six hours at 37° C. in 5% CO₂. To eliminate cells which had not migrated through the filter, the upper side of the filter was scraped clean with a cotton bud. Subsequently, the filter was removed and fixed in methanol and subsequently coloured with haematoxylin and eosin. The cell count in five randomly selected regions was determined at a 200× magnification using a phase contrast microscope (Leica microsystems GmbH, Welzlar, Germany). HLE-B3 cells of the same passage, incubated with the same volume of PBS without the addition of APCs, were used as a control.

HLE-B3 migration was effectively inhibited by APCs (see FIG. 5).

Close to the IC₅₀ concentration thereof, APCs were able to induce a reduction of the cell migration to 43.1% (0.1 mM) of the cells by comparison with the controls. An APC concentration of 1 mM was able to induce a maximum migration inhibition of 95%.

### Example 3

### Effectiveness of an Intraocular Lens Coated with APCs in the Prophylaxis of Aftercataract Intraocular lenses are coated with APC, as explained in Example 1,m and kept sterile at room temperature. Human lens epithelial cells are cultivated in petri dishes under standard cell culture conditions, as in Example 2.1. When semiconfluence of the cells on the respective plate is achieved, an APC-coated intraocular lens is laid in the centre of the plate. Non-APC-coated intraocular lenses are used as a control. After 3, 5 and 7 days, the lenses are removed and the cell count of the cells left in the cell culture dishes are determined, as described in 2.1, by cell counters and by the MTT test. A considerably reduced number of lens epithelial cells were found in the cell culture dishes into which an APC-coated intraocular lens had been introduced.

FIG. 6 (bar chart of the inhibition of human lens epithelial cell proliferation by oleylphosphocholine-coated intraocular lenses) shows that intraocular lenses of various materials (hydrophobic acrylates, hydrophilic acrylates and silicones) having different haptic designs and optical diameters, coated with alkylphosphocholines (OIPC), can inhibit the cell growth of lens epithelial cells which are already proliferating.

A tetrazolium reduction assay [MIT (3-[4,5-dimethylthiazol-2-yl]-2,5-diphenyltetrazolium bromide] (Sigma-Aldrich) was carried out to determine the cell count. The intraocular lenses were removed, and the cells were washed with PBS and incubated with the MTT reagent for 1 hour at 37° C. Only vital cells metabolise the solution. Subsequently, the cell membrane was dissolved with DMSO (dimethylsulphoxide) and the optical thickness was measured in the ELISA reader at 550 nm. In this case, the optical thickness correlates with the number of proliferated cells.

In FIG. 7 (bar chart of the inhibition of human lens epithelial cell proliferation by erucylphosphohomocholine-coated intraocular lenses), this is also shown for intraocular lenses of different materials (hydrophobic acrylates, hydrophilic acrylates and silicones), having different haptic designs and optical diameters, which have been coated with ErPC₃.

### Example 4

### Safety Profile of the Alkylphosphocholines Substance Group on the Model of Isolated Perfused Bovine Retina

The safety of alkylphosphocholines (APCs) was analysed in the ex vivo model of the perfused bovine retina. For this purpose, preparations of bovine retina were perfused with an oxygen-preequilibrated standard solution. The electroretinogram (ERG) was taken using Ag/AgCl electrodes. After stable b-wave amplitudes were recorded, alkylphosphocholine was added to the nutrient solution at concentrations of 0.25 μM, 2.5 μM and 25 μM. To determine the effects of alkylphosphocholines on the photoreceptor function, a test series, in which the effects of alkylphosphocholine on the a-wave amplitude were assessed, was carried out at the same concentrations For this purpose, aspartate was added to the nutrient solution at a concentration of 1 μM to obtain stable wave amplitudes. Subsequently, alkylphosphocholine was added to the nutrient solution at the same concentrations. The ERG amplitudes were observed for 75 minutes.

No reductions in the a-wave and b-wave amplitudes were observed in the test series at the end of the treatment with alkylphosphocholine. No differences between the ERG amplitudes before and after the treatment with alkylphosphocholine were established.

### Example 5

### Coating Intraocular Lenses with APCs

In a 6-well plate, well 1 is filled with 1.5 ml APC parent solution and well 3 is filled with 1.5 ml of a physiological saline solution. An intraocular lens (IOL) is placed in the APC parent solution (10 mM APC in physiological saline solution) in well 1 and incubated overnight at room temperature. Subsequently, the intraocular lens is removed and laid in the empty well 2 and incubated for 3 hours at 4° C. Subsequently, the intraocular lens is transferred into well 3 (physiological saline solution) and incubated for 1 hour at 37° C. After the removal of the intraocular lens, the 6-well coating plate is wrapped in foil and kept at 4° C.

The APC-coated intraocular lens (APC-IOL) is laid on proliferating HLE-B3 cells (6-well plate, approx. 60% confluence) (volume 1.5 ml MEM/5% FCS). An intraocular lens of the same type without APC coating was used as a control.

Every two days, the cells were analysed for growth/encrustation of the IOL or migration onto the IOL and photodocumented.

After 10 to 14 days, the IOLs are removed and the cells are released, and the cell count is determined and compared. The cell count was determined in a Neubauer counting chamber or in a Casy cell counter.

### Example 6

### Prevention of Aftercataract Formation on Intraocular Lenses Coated with Alkylphosphocholines

In this example, the capacity of APC-coated intraocular lenses for inhibiting proliferation of human lens epithelial cells was analysed. For this purpose, intraocular lenses (IOLs) of different designs (three-piece and single-piece IOLs) were introduced into a reserve APC solution, washed with PBS and dried overnight. Uncoated IOLs having the same designs were used as a control. The IOLs were each placed in a well of a 24-well plate, said wells comprising proliferating human lens epithelial cells (HLE-B3), and cultivated under standard cell culture conditions for three days. Subsequently, the MTT test was carried out and the cell count per well was calculated. On days 2 and 3, the cells around and below the intraocular lens were photodocumented (FIG. 8). It is found that when the alkylphosphocholine-coated intraocular lens is introduced into a culture, it leads to inhibition of the cellular propagation and the growth of the cells both around and below the intraocular lens. The effect strengthens over time (increase in the effect from 48 to 72 hours after introducing the alkylphosphocholine-coated intraocular lenses into the cell culture).

The analyses showed that the proliferation of human lens epithelial cells was inhibited by APC-coated intraocular lenses. The results, given in percent based on the controls, were as follows:

39%±25 for single-piece IOLs

and

79%±10 for three-piece IOLs.

This showed that the cell proliferation on single-piece IOLs was inhibited more effectively than the cell proliferation on three-piece IOLs.

It is found that single-piece intraocular lenses in particular benefit from the alkylphosphocholine coating.

The tests showed that APCs are adapted coating agents for intraocular lenses which can significantly inhibit the proliferation of human lens epithelial cells. Lenses of this type can thus be used for the pharmacological prophylaxis of aftercataract formation (posterior capsule opacification).

1-18. (canceled)

19. A medical material comprising a phosphocholine compound of formula (1) 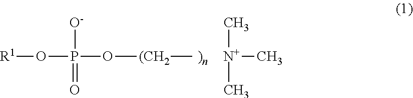
wherein R¹ is a hydrocarbon radical optionally comprising heteroatoms and n is an integer from 1 to 5.

20. The medical material of claim 19, wherein the phosphocholine compound is coated on the medical material.

21. The medical material of claim 19, wherein the phosphocholine compound is mixed into a base material of the medical material.

22. The medical material of claim 19, wherein the medical material is at least part of an ophthalmological implant.

23. The medical material of claim 19, wherein the ophthalmological implant is at least part of an intraocular lens.

24. The medical material of claim 19, wherein the medical material is at least part of a medical implant.

25. The medical material of claim 19, wherein the medical material is at least part of a stent, a heart valve, a permanent catheter, a prosthesis, an implant, a suturing material or a contact lens.

26. The medical material of claim 19, wherein the medical material is at least part of a refractive, deformable polymer.

27. The medical material of claim 19, wherein the medical material is selected from the group consisting of hydrophobic acrylates, hydrophilic acrylates and silicone.

28. The medical material of claim 19, wherein R¹ is a hydrocarbon radical selected from the group consisting of a C₁₆-C₂₄ hydrocarbon radical, —CH₂ (CH₂)ₓ—CH₂O—R⁴ and —CH₂—CH[—O—(CH₂)_{y}—H]—CH₂—O—R⁴,
wherein
R⁴ is a hydrocarbon radical, in particular a C₁₆-C₂₄ hydrocarbon radical,
x represents an integer from 0 to 4, and
y represents an integer from 1 to 3.

29. The medical material of claim 19, wherein that the phosphocholine compound is erucylphosphocholine, erucylhomophosphocholine or oleylphosphocholine.

30. A method for improving surface properties and/or biocompatibility of a medical material, such as an implant, comprising employing the medical material of claim 19.

31. A method for improving wound healing reactions or for reducing foreign-body reactions to a medical material, such as an implant, comprising employing the medical material of claim 19.

32. A method for the prophylaxis of aftercataract, reduction of the incidence of aftercataract or prevention of aftercataract comprising employing the medical material of claim 19 in an ophthalmological implant.

33. A method for producing a medical material comprising employing a phosphocholine compound of formula (I) in the medical material 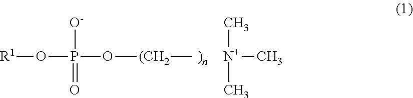
wherein R¹ is a hydrocarbon radical optionally comprising heteroatoms and n is an integer from 1 to 5.

34. The method of claim 33, wherein the medical material is an ophthalmological implant.

35. A method for the prophylaxis, prevention or treatment of an ophthalmological disorder comprising employing a phosphocholine compound of formula (1) in an ophthalmological implant, 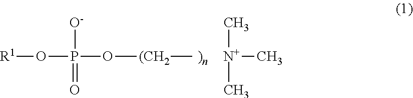
wherein R¹ is a hydrocarbon radical optionally comprising heteroatoms and n is an integer from 1 to 5.

36. The method of claim 35, wherein the phosphocholine compound is erucylphosphocholine, erucylhomophosphocholine or oleylphosphocholine.

37. The method of claim 35, wherein the ophthalmological disorder is an aftercataract.
